(19) United States
(12) Patent Application Publication
(10) Pub. No.: US 2023/0018373 A1
(43) Pub. Date: Jan. 19, 2023

(54) AUTOMATED PRODUCTION OF VIRAL VECTORS

(71) Applicants: LONZA WALKERSVILLE, INC., Walkersville, MD (US); LONZA SALES AG, Basel (CH)

(72) Inventors: Evlampia DIMITRIADOU, Slough, Berkshire (GB); Matthew HEWITT, Walkersville, MD (US); Andrew RACHER, Slough, Berkshire (GB)

(21) Appl. No.: 17/757,345

(22) PCT Filed: Dec. 18, 2020
(86) PCT No.: PCT/US2020/066006

§ 371 Date: Jun. 14, 2022

Related U.S. Application Data
(60) Provisional application No. 62/950,177, filed on Dec. 19, 2019.

(51) Int. Cl. C12N 15/86; C12M 1/34; G16B 99/00

(52) U.S. Cl. CPC C12N 15/86; C12M 41/12; C12M 41/26; C12M 41/32; C12M 41/34; C12M 41/36; G16B 99/00; C12N 2710/14143; C12N 2710/14151; C12N 2740/13043; C12N 2740/13051; C12N 2750/14143; C12N 2750/14151

(57) ABSTRACT

The present disclosure provides an automated method of producing viral vectors, utilizing engineered viral vector-producing cell lines, or packaging cells, within a fully-enclosed cell engineering system. Exemplary viral vectors that can be produced include lentivirus vectors, adeno-associated virus vectors, baculovirus vectors and retrovirus vectors.

### FIELD OF THE INVENTION

The present disclosure provides an automated method of producing viral vectors, utilizing engineered viral vector-producing cell lines, or packaging cells, within a fully-enclosed cell engineering system. Exemplary viral vectors that can be produced include lentivirus vectors, adeno-associated virus vectors, baculovirus vectors and retrovirus vectors.

### BACKGROUND OF THE INVENTION

Viral vectors are critically important as both basic research tools and for use in gene therapeutics. For example, the safety profile and long-term expression capacity make adeno-associated virus (AAV) an excellent viral vector for gene therapy in humans. Lentiviral vectors, similarly, are one of the most commonly used delivery methods in the field of gene and cell therapy. However, the traditional means of producing most viral vectors is expensive, time-consuming, and cumbersome. Furthermore, vector yield from methods that rely on bridging platforms (such as AAVs) or muliple transient transfections (such as lentivirus) may be too low or require too much plasmid DNA to be feasible for most therapeutic applications. In addition, for small scale viral vector production, a large batch process may not be required or desirable.

The benefits of automation of production of viral vectors include labor time savings associated with using automation as well as improved product consistency, decreased room classification, decreased clean room footprint, decreased training complexities, and improved scale-up and tracking logistics. Furthermore, software can be used to streamline the documentation processes by using automatically generated electronic batch records to provide a history of all processing equipment, reagents, operator identification, in-process sensor data, and so forth.

An automated, self-contained system in which engineered mammalian cells optimally produce viral vectors would revolutionize the field of gene therapy. There is an urgent need for technology that would allow control of viral production for large or small scale volume production, provide reproducible and stable results, while simultaneously limiting contamination and reducing cost.

### SUMMARY OF THE INVENTION

In some embodiments, provided herein is a method for automated production of a viral vector, comprising: introducing an engineered viral producer cell into a fully enclosed cell engineering system; transducing the engineered viral producer cell with a vector encoding a gene of interest to produce a transduced viral producer cell; expanding the transduced viral producer cell and producing the viral vector within the transduced viral producer cell; transferring the expanded producer cell to a downstream processing module; and isolating the viral vector; and purifying the viral vector, wherein (a) through (e) are performed in a closed and automated process.

Also provided herein is a method for automated production of a viral vector, comprising: introducing a packaging cell into a fully enclosed cell engineering system; transducing the packaging cell with one or more vectors encoding a viral helper gene, a viral packing gene, and a gene of interest to produce a transduced cell; expanding the transduced cell and producing the viral vector within the transduced cell; transferring the expanded cell to a downstream processing module; and isolating the viral vector; and purifying the viral vector, wherein (a) through (e) are performed in a closed and automated process.

### BRIEF DESCRIPTION OF THE DRAWINGS

FIG. 1 shows a flow diagram for the automated production of a viral vector in accordance with embodiments hereof

FIG. 2 shows a closed and automated cell engineering system as described in embodiments herein.

FIG. 3 shows a lab space containing exemplary closed and automated cell engineering systems as described in embodiments herein.

FIG. 4 shows a diagram of a viral vector production process that can be performed in a cassette of a closed and automated system as described in embodiments herein.

FIG. 5 shows a flow diagram of a process within an automated cell engineering system as described herein.

FIG. 6 shows a block diagram of downstream processing in accordance with embodiments hereof.

FIG. 7 shows a flow diagram of downstream processing in accordance with embodiments hereof.

FIGS. 8A-8D show components of a downstream processing module in accordance with embodiments hereof.

FIG. 9 shows an exemplary software control design for use with a downstream processing module in accordance with embodiments hereof.

FIGS. 10A and 10B show two views of a downstream processing module in accordance with embodiments hereof.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the invention. Furthermore, compositions, systems, cells, and/or nucleic acids of the invention can be used to achieve any of the methods as described herein.

In embodiments, provided herein is a method for automated production of a viral vector. The automated methods described herein suitably are performed in a closed an automated process.

A “viral vector,” as produced by the methods described herein, refers to a product virus that can be used to introduce a nucleic acid molecule into a cell in vitro, in vivo, or ex vivo, suitably for a therapeutic or industrial purpose. Viral vectors produced by the various methods described herein can be harvested or isolated, and stored until a final desired application.

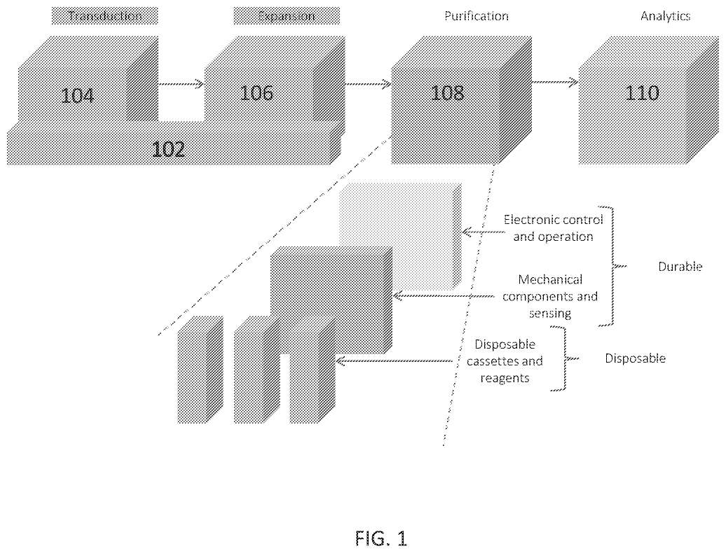

FIG. 1 shows a block diagram of the flow of the automated production processes described herein.

Suitably, the methods described herein include introducing an engineered viral producer cell into a fully enclosed cell engineering system. As referred to herein, an “engineered viral producer cell” utilized in the methods is a cell that suitably includes one or more nucleic acid molecules encoding helper genes or expression systems that allow for production of viral vectors.

As referred to herein, the word “introducing” can mean adding the engineered viral producer cell to one of a plurality of chambers, or can indicate the presence of the engineered viral producer cell within the cassette prior to beginning the method.

In embodiments, the methods described herein are configured to perform several rounds of one or more of feeding, washing and monitoring of the engineered viral producer cell. These various activities can be performed in any order and can be performed alone or in combination with another activity. In embodiments, concentrating of the cells comprises centrifugation, supernatant removal following sedimentation, or filtration. Suitably, the optimization process further includes adjusting parameters of the centrifugation or filtration, suitably in a self-adjusting process.

The methods described herein suitably further include transducing the engineered viral producer cell with a vector encoding a gene of interest to produce a transduced viral producer cell.

As referred herein, “transduction” or “transducing” means the introduction of an exogenous nucleic acid molecule, including a vector, into a cell. A “transduced” cell comprises an exogenous nucleic acid molecule inside the cell and induces a phenotypic change in the cell. The transduced nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to as “recombinant,” “transduced,” “transfected,” or “transgenic” organisms. A number of transduction and transfection techniques are generally known in the art. See, e.g., Graham et al., Virology, 52:456 (1973); Sambrook et al., Molecular Cloning, a laboratory manual, Cold Spring Harbor Laboratories, New York (1989); Davis et al., Basic Methods in Molecular Biology, Elsevier (1986); and Chu et al., Gene 13:197 (1981). Transduction can include the use of a transfection system such as a liposome, lipid-based, or polymer-based system, and can also include the use of mechanical transfection such as gene guns, electroporation, etc.

As used herein, a “vector” or “expression vector” is a replicon, such as a plasmid, phage, virus, or cosmid, to which a nucleic acid molecule described herein may be attached to bring about the replication and/or expression of the attached nucleic acid molecule in a cell. “Vector” includes episomal (e.g., plasmids) and non-episomal vectors. The term “vector” includes both viral and non-viral means for introducing a nucleic acid molecule into a cell in vitro, in vivo, or ex vivo. The term vector may include synthetic vectors. Vectors may be introduced into the desired host cells by well-known methods, including, but not limited to, transfection, transduction, cell fusion, and lipofection. Vectors can comprise various regulatory elements including promoters.

A “gene” refers to an assembly of nucleotides that encode a polypeptide and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence. In some embodiments, genes are integrated with multiple copies. In some embodiments, genes are integrated at predefined copy numbers.

As referred to herein, the term “gene of interest” or “GOT” is used to describe a heterologous gene. As referred to herein, the term “heterologous gene” or “HG” as it relates to nucleic acid sequences such as a coding sequence or a control sequence, denotes a nucleic acid sequence, e.g. a gene, that is not normally joined together, and/or are not normally associated with a particular cell. In some embodiments, a heterologous gene is a construct where the coding sequence itself is not found in nature (e.g., synthetic sequences having codons different from the native gene). Allelic variation or naturally occurring mutational events do not give rise to heterologous DNA, as used herein.

Suitably, the gene of interest that is transduced into the viral producer cells is a gene of therapeutic interest. As used herein a “gene of therapeutic interest” refers to any functionally relevant nucleotide sequence. Thus, the gene of therapeutic interest of the present disclosure can comprise any desired gene that encodes a protein that is defective or missing from a target cell genome or that encodes a non-native protein having a desired biological or therapeutic effect (e.g., an antiviral function), or the sequence can correspond to a molecule having an antisense or ribozyme function. Representative (non-limiting) examples of suitable genes of therapeutic interest include those used for the treatment of inflammatory diseases, autoimmune, chronic and infectious diseases, including such disorders as AIDS, cancer, neurological diseases, cardiovascular disease, hypercholestemia; various blood disorders including various anemias, thalassemias and hemophilia; genetic defects such as cystic fibrosis, Gaucher's Disease, adenosine deaminase (ADA) deficiency, emphysema, etc. Several antisense oligonucleotides (e.g., short oligonucleotides complementary to sequences around the translational initiation site (AUG codon) of an mRNA) that are useful in antisense therapy for cancer and for viral diseases have been described in the art and are also examples of suitable genes of therapeutic interest.

In exemplary embodiments, the methods described herein, and thus the gene of therapeutic interest, are useful in producing viral vectors for applications in ultra-rare diseases. Such diseases may not require a significant amount of viral vector (as it may be a single or a few patients, or 10's of patients, or 100's of patients, for treatment), but sterility, reproducibility and process control are extremely critical in such applications. The methods described herein utilizing a closed an automated system allow for the desire level of control of the production.

In embodiments, the methods further comprise expanding the transduced viral producer cell and producing the viral vector within the transduced viral producer cell. As described herein, the methods of expanding the transduced viral producer cells suitably include at least one or more of feeding, washing, and monitoring. “Expanding” a transduced viral producer cell refers to various methods that allow the cells to grow until they reach a pre-defined, desired culture size. The pre-defined culture size may include a sufficient number of cells that allow for the production of a suitable or desirable number of viral vectors. In some embodiments, the number of viral producer cells is about 10⁵ cells, about 10⁶ cells, about 10⁷ cells, about 10⁸ cells, about 10⁹ cells, or about 10¹⁰ cells.

As illustrated in FIG. 1, suitably the transduction 104 and expansion 106, take place within a full enclosed cell engineering system 102. Suitably, these fully enclosed cell engineering systems are automated systems.

As described herein, “a fully enclosed cell engineering system” refers to a closed system, suitably including a plurality of chambers, and wherein each of steps of the various methods described herein are performed in the same or a different chamber of the plurality of chambers of the cell engineering system. Suitably, each of the various cells, vectors and cell culture medium, are contained in a different chamber of the plurality of the chambers prior to starting the method. The cell engineering systems suitably include one or more chambers maintained at a temperature for growing cells (e.g., at about 37° C.) and at least one of the plurality of chambers is maintained at a refrigerated temperature (e.g., at about 4-8° C.). “Fully enclosed” suitably refers to the plurality of chambers being interconnected, including via various tubing or other fluidly connected pathways and connections, to maintain the cleanness, and suitably sterility, of the fully enclosed systems.

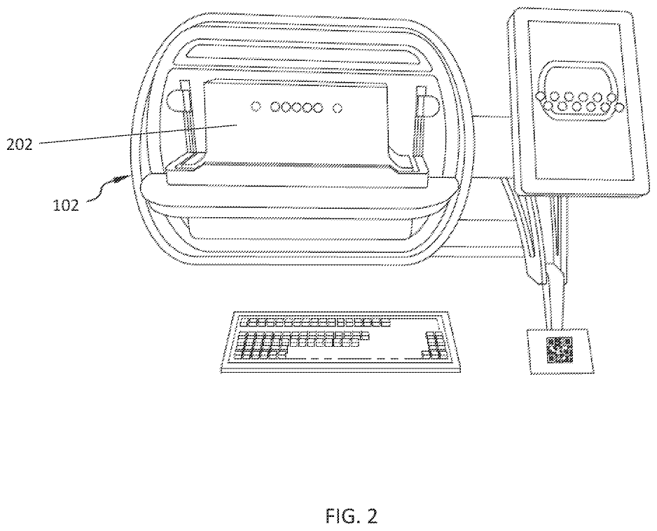

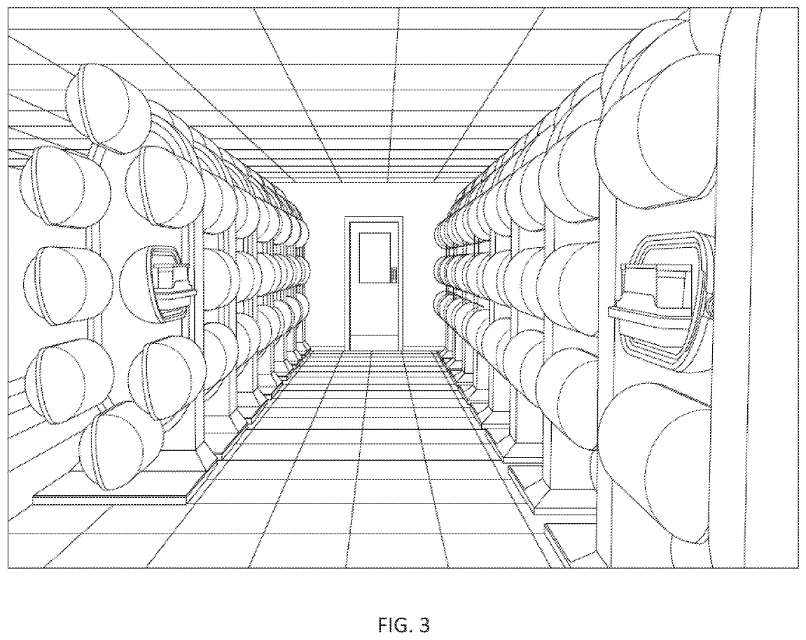

As described herein, in embodiments, the methods provided utilize the COCOON platform (Octane Biotech (Kingston, ON)), which integrates multiple unit operations in a single turnkey platform. To provide efficient and effective automation translation, the methods described utilize the concept of application-specific/sponsor-specific disposable cassettes that combine multiple unit operations—all focused on the core requirements of the viral vector product. Exemplary fully enclosed cell engineering systems are described in Published U.S. Patent Application No. 2019-0169572, the disclosure of which is incorporated by reference herein in its entirety. An exemplary fully enclosed cell engineering system 102 useful in the methods described herein is shown in FIG. 2. FIG. 3 shows a lab space containing exemplary fully enclosed cell engineering systems 102 useful for producing viral vectors as described in embodiments herein in a high throughput arrangement. In embodiments, each of the closed and automated systems can be producing a separate and unique viral vector.

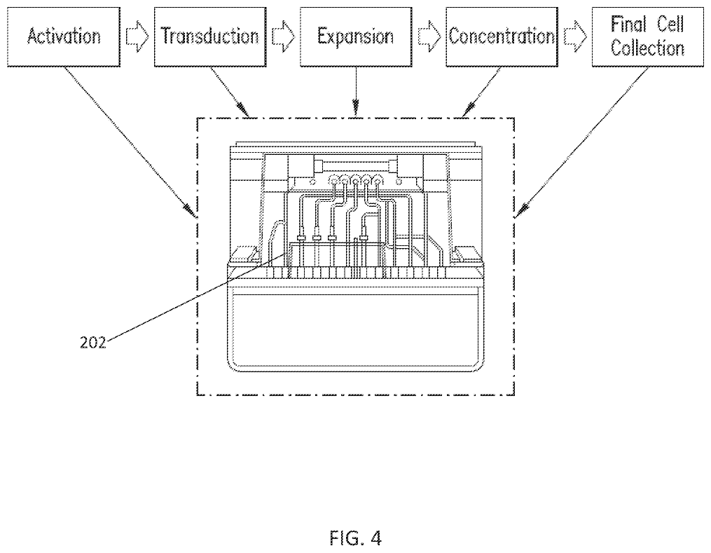

In embodiments, the transducing and expanding described herein take place in a cassette 202 of a fully enclosed cell engineering system 102 (see FIG. 2 and FIG. 4). Cassette 202 can include a low temperature chamber, for storage of a cell culture media; a high temperature chamber for carrying out processes involved in producing a viral vector, wherein the high temperature chamber is separated from the low temperature chamber by a thermal barrier, the high temperature chamber including a cell culture chamber; and one or more fluidics pathways connected to the cell culture chamber, wherein the fluidics pathways provide recirculation, removal of waste and homogenous gas exchange and distribution of nutrients to the cell culture chamber without disturbing cells within the cell culture chamber. FIG. 4 shows a flow diagram of elements of a viral vector production process that can be performed in a cassette 202 as described in embodiments herein.

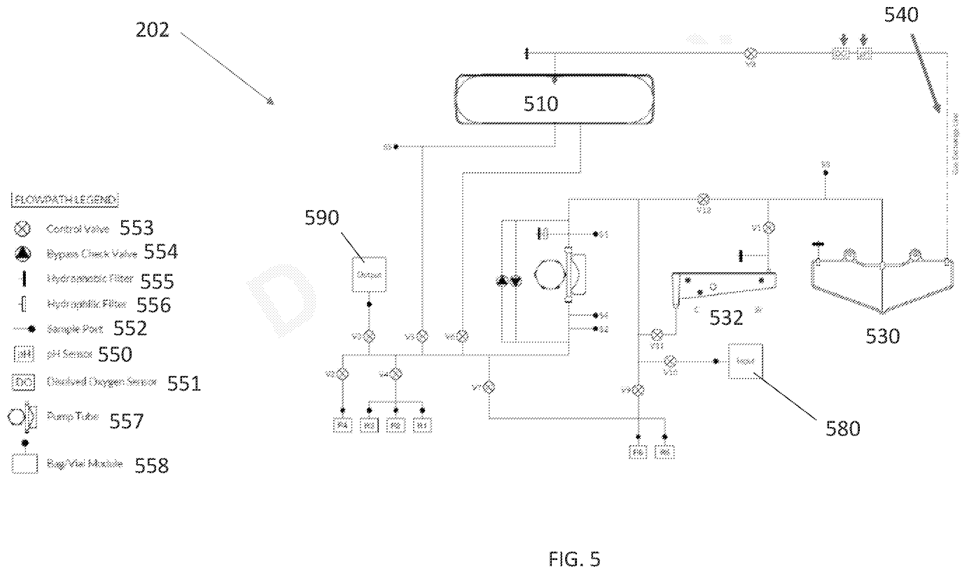

FIG. 5 shows a flow diagram of various components of a cassette 202. FIG. 5 shows a schematic illustrating the connection between cell culture chamber 510, and satellite volume 530. Also illustrated in FIG. 5 are the positioning of various sensors (e.g., pH sensor 550, dissolved oxygen sensor 551), as well as sampling/sample ports 552 and various valves (control valves 553, bypass check valves 554), as well as one or more fluidic pathways 540, suitably comprising a silicone-based tubing component, connecting the components. As described herein, use of a silicone-based tubing component allows oxygenation through the tubing component to facilitate gas transfer and optimal oxygenation for the cell culture. Also show in FIG. 5 is the use of one or more hydrophobic filters 555 or hydrophilic filters 556, in the flow path of the cassette, along with pump tube 557 and bag/valve module 558. FIG. 5 also illustrates exemplary positions for input 580, where an engineered viral producer cell (or a packaging cell) can be introduced into the cassette 202, as well as output 590, where the expanded producer cell lines (or expanded packaging cells) can be withdrawn and transferred to a downstream processing module 108.

As shown in FIG. 1, following the expansion, the expanded producer cell line (or expanded packing cell line) is transferred to downstream processing module 108. As used herein “transferred” suitably refers to the direct connection between the fully enclosed cell engineering system 102 and downstream processing module 108, for example by connecting the output 590 of the system 102 to an input of downstream processing module 108 so as to maintain a closed system and process. As described herein, all of the elements of the automated production method (from transduction, expansion, isolation to purification) are suitably performed in a closed and automated process. The term “closed” process suitably refers to the use of a cartridge and other contained systems that do not allow for interaction with the external environment (unless desired), with a direct connection to a downstream processing module 108, so as to maintain a sterile process. The “automated” process or “automation” of the process refers to the control of one or more process described herein by an external control, including a microprocessor, to monitor and change the parameters based on a defined, or pre-set set of circumstances or desired characteristics.

Suitably, the downstream processing module 108 carries out processes such as isolation (or isolating) of the viral vector and purification (or purifying) of the viral vector. As shown in FIG. 1, downstream processing module 108 is suitably a compact, automated, easily configured and changed processing module. Downstream processing module 108 suitably includes electronic control and operation, and mechanical components and sensing. Suitably, the electronic control and the mechanical components are durable components, in that they do not need to be readily replaced with each viral production process. Downstream processing module 108 also suitably includes disposable cassettes and reagents that are replaceable (and suitably replaced) following each viral production (or at least between different types of virus being produced).

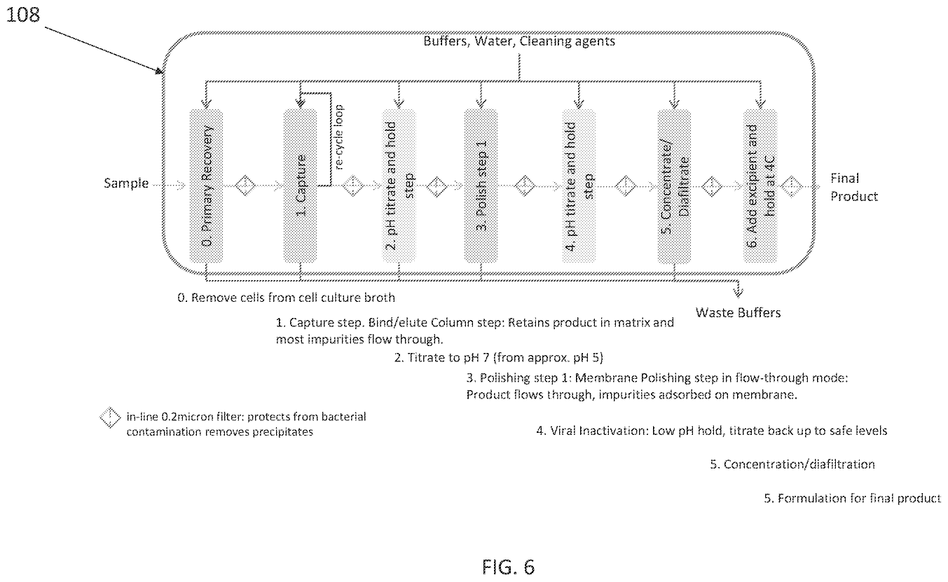

FIG. 6 shows an exemplary block diagram of suitable activities that take place within downstream processing module 108. As shown, in embodiments, an expanded cell product (sample) is first subjected to primary recovery to remove cells from a cell culture broth. Cells are then suitably lysed to expose the product viral vectors, suitably by mechanical (e.g., beads, shaking, etc.) or chemical means (e.g., lysis buffers, detergents, etc.). A capture step is then used to isolate the viral vectors. This isolating is suitably a bind/elute column step in which the product (viral vectors) remain in the matrix and impurities flow through. Suitable column conditions and media are known in the art. A retronectin or fibronectin coated surface can also be used. This capture step can be repeated as many times as desired until the total amount of virus is collected. This isolation can also include the use of sedimentation columns, as well as chromatography columns and various affinity columns, including sepharose columns that can include functionalized surfaces. Following the initial capture, the solution is suitably titrated from 5 up to about 7.

A polishing step is then carried out, for example a membrane polishing step in a flow-through mode, to purify the viral vector. In such a polishing, impurities are absorbed on the membrane and the desired product (viral vector) flows through. An exemplary polishing step utilizes a strong ion exchanger such as a SARTOBIND® Q ion exchanger (SARTORIUS®, Gottingen, Germany). This polishing step removes undesired viruses, DNA, host cell proteins, leached protein A and endotoxins. Exemplary buffers and conditions for carrying out the polishing step are known in the art.

Following the polishing, a pH titration and hold step are suitably carried out. This suitably involves dropping pH down to below pH 6 or pH 5, holding for a desired time, and then titrating the pH back up to about pH 7. The processing suitably further includes concentrating or diafiltering the viral particles to achieve the desired concentration.

Following the concentration, the viral vector product can then be formulated for final product. This can include the addition of various excipients (e.g., salts, buffers, osmolarity adjusting agents), as well as different media, etc. The viral vector product is then suitably held at a reduced temperature (e.g. about 4-8° C.) until it can be either administered to patient, or packaged/shipped or stored.

Between the various elements of the downstream processing module, an in line 0.2 micron filter is suitably used to protect from bacterial contamination and to remove precipitates.

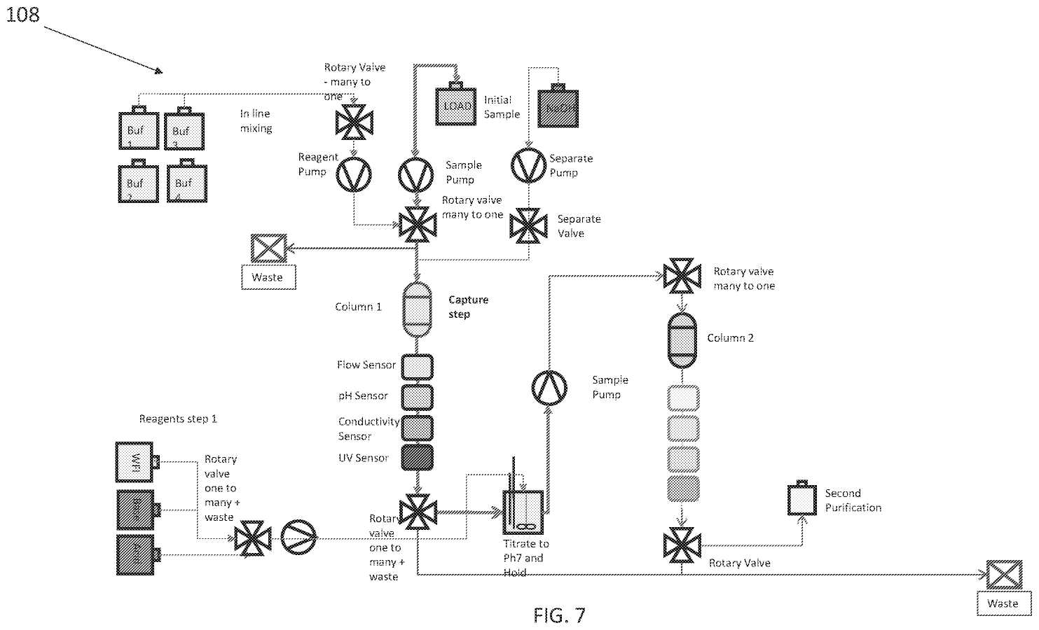

FIG. 7 shows an exemplary flow diagram and exemplary components of a downstream processing module 108 as described herein. As shown, the downstream processing module suitably includes various buffers and reagents, as well as columns, that can be replaced for each run or for a different viral vector system, as well as components that are not replaced, e.g., pumps, valves, control systems, etc.

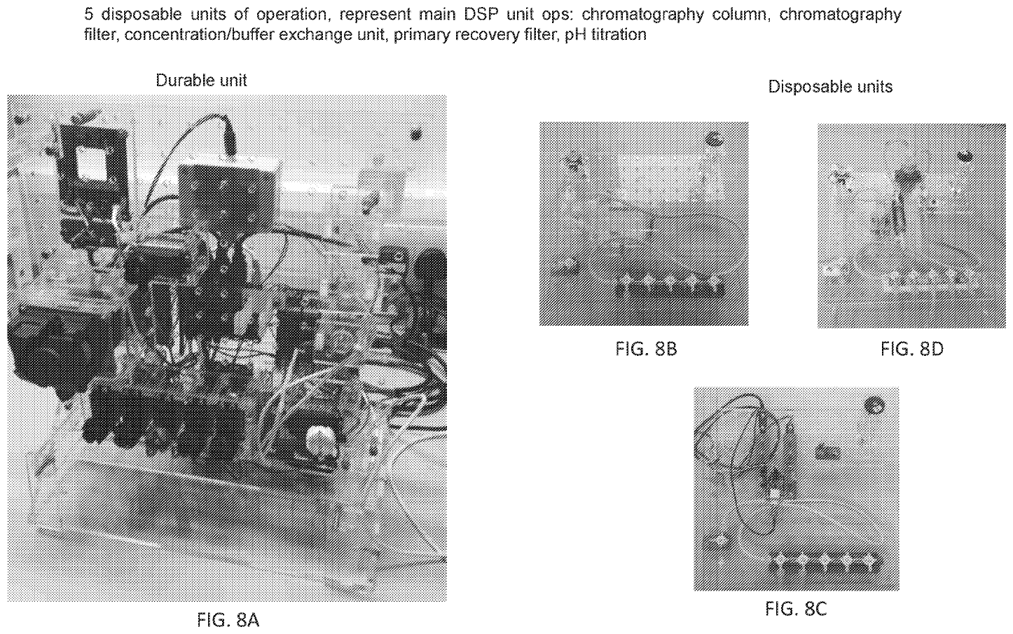

FIG. 8A shows an exemplary downstream processing module 108. FIGS. 8B-8D show disposable/replaceable elements that contain the different buffers and columns that can be switched out.

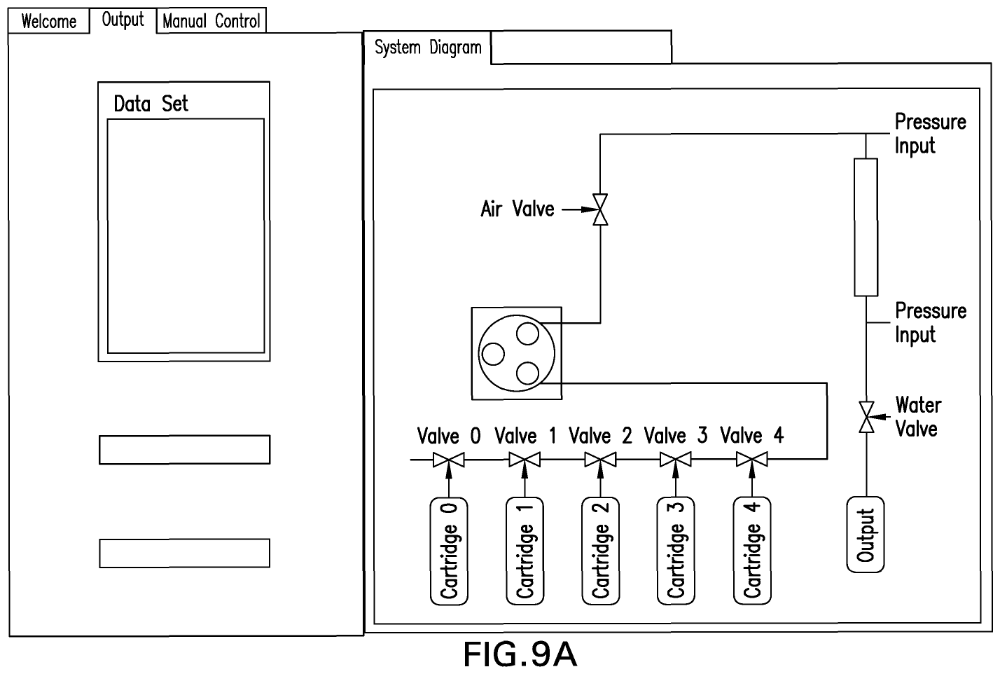

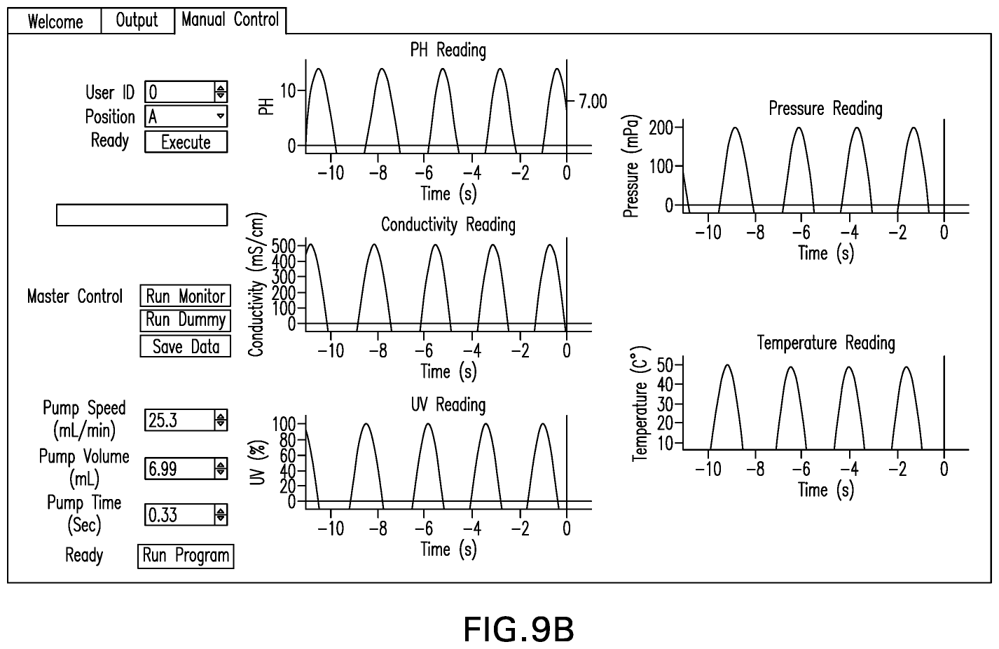

FIG. 9 shows an exemplary computer control set-up for a downstream processing module 108, showing an interface to control the various valves, pumps, etc., as well as monitoring of the systems. Also shown is a mock output, illustrating various elements that can be monitored, such as pH, conductivity, UV, temperature, pressure, etc.

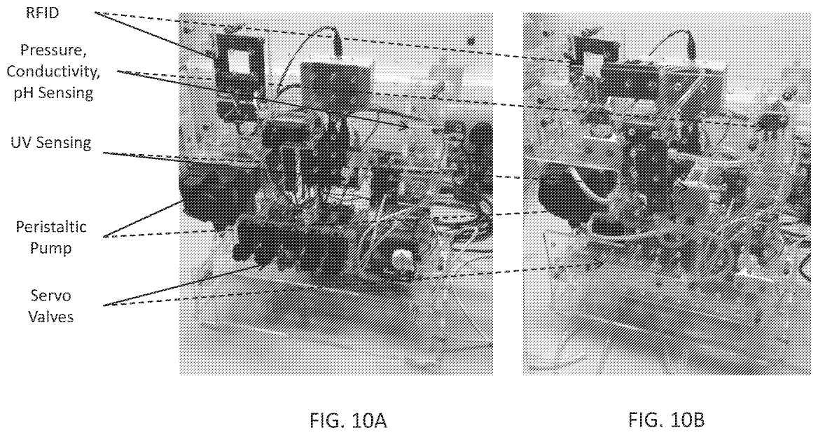

As shown in FIGS. 10A and 10B, downstream processing module 108 can include components such as a radio frequency identification (RFID) reader to correlate a user or a specific sample with a specific module. Also shown are pressure, conductivity and pH sensors, UV sensors, peristaltic pumps and servo valves.

As illustrated in FIG. 1, following the downstream processing module 108, the viral vector product can be passed on for further analytics 110, including measurement of viral titer, activity levels, contamination, etc.

In embodiments, the closed and automated process is a self-adjusting process, that is one that does not require input from an external (human) user and is able, via various computer programs and conditions, to determine the required modifications to a cell culture or other characteristics to optimize the automated process. In embodiments, the closed and automated process includes monitoring with one or more of a temperature sensor, a pH sensor, a glucose sensor, a lactose sensor, an oxygen sensor, a carbon dioxide sensor, and an optical density sensor. As described herein, the use of these various sensors in the fully enclosed cell engineering system occurs at various times and locations within the system and work together in concert to provide the optimization. For example, the closed and automated process can adjust (e.g., raise or lower) one or more of a temperature, a pH level, a glucose level, a lactose level, an oxygen level, a carbon dioxide level, and an optical density of the viral-producing cell culture, based on the monitoring.

The automated process can also be based on the unique characteristics of the starting cell population, including for example, the total cell number, the source of the cells, the density of the cells, the age of the cells, etc. These starting cell population characteristics can be input into a computer control system prior to beginning the automated methods, upon which the system will make various initial modifications to optimize the methods, e.g., lactose, oxygen and carbon dioxide concentration, flow rates, incubation times, pH, etc. Alternatively, the monitoring of cell processes enables the automated characterization of the progress of the cell culture sequence from the starting population to enable case-by-case adjustment of conditions for optimized final cell culture properties.

In further embodiments, the cell engineering system recirculates nutrients, waste, released cytokines, and/or dissolved gasses during the various method processes. This recirculation helps aid in the production of the desired viral vectors. Other mechanisms for optimizing the production of the viral vectors include modifying and controlling the flow rate of the media provided to the cells. As the cells begin to grow, the circulation rate of the media provided is increased, which improves gas exchange and allows oxygen and carbon dioxide to either enter or leave the cell culture, depending on the conditions of the cells and the requirements at the time.

In additional embodiments, the methods and systems described herein can also be used with a transient transfection system. In such embodiments, a method for automated production of a viral vector includes: introducing a packaging cell into a fully enclosed cell engineering system; transducing the packaging cell with one or more vectors encoding a viral helper gene, a viral packing gene, and a gene of interest to produce a transduced cell; expanding the transduced cell and producing the viral vector within the transduced cell; transferring the expanded producer cell to a downstream processing module; and isolating the viral vector; and purifying the viral vector, wherein the elements of the method are performed in a closed and automated process.

In embodiments that utilize transient transfection, a packaging cell can be utilized. As used herein, a “packaging cell” refers to a cell that does not have integrated into its genome one or more viral helper and/or packaging genes, but instead these genes are added via transfection to produce a transiently transfected cell.

In embodiments, the engineered viral producer cell or the packaging cell that is utilized in the automated methods is a mammalian cell. As used herein, the term “mammalian cell” includes cells from any member of the order Mammalia, such as, for example, human cells, mouse cells, rat cells, monkey cells, hamster cells, and the like. In some embodiments, the cell is a mouse cell, a human cell, a Chinese hamster ovary (CHO) cell, a CHOK1 cell, a CHO-DXB11 cell, a CHO-DG44 cell, a CHOK1SV cell including all variants (e.g. POTELLIGENT®, Lonza, Slough, UK), a CHOK1SV GS-KO (glutamine synthetase knockout) cell including all variants (e.g., XCEEDTM Lonza, Slough, UK). Exemplary human cells include human embryonic kidney (HEK) cells, such as HEK293, HEK293T, a HeLa cell, or a HT1080 cell.

Mammalian cells include mammalian cell cultures which can be either adherent cultures or suspension cultures. Adherent cultures refer to cells that are grown on a substrate surface, for example a plastic surface, plate, dish or other suitable cell culture growth platform, and may be anchorage dependent. Suspension cultures refer to cells that can be maintained in, for example, culture flasks or large suspension vats, which allows for a large surface area for gas and nutrient exchange. Suspension cell cultures often utilize a stirring or agitation mechanism to provide appropriate mixing. Media and conditions for maintaining cells in suspension are generally known in the art. An exemplary suspension cell culture includes human HEK293 clonal cells.

In embodiments, the methods of production of a viral vector provided herein produce an adeno-associated virus (AAV) vector.

As used herein, the term “adeno-associated virus (AAV) vector” refers to a small sized, replicative-defective, nonenveloped virus containing a single stranded DNA of the family Parvoviridae and the genus Dependoparvovirus. Over 10 adeno-associated virus serotypes have been identified so far, with serotype AAV2 being the best characterized. Other non-limiting examples of AAV serotypes are ANC80, AAV1, AAV3, AAV4, AAV5, AAV6, AAV7, AAV8, AAV9, AAV10, and AAV11. In addition to these serotypes, AAV pseudotypes have been developed. An AAV pseudotype contains the capsid of a first serotype and the genome of a second serotype (e.g. the pseudotype AAV2/5 would correspond to an AAV with the genome of serotype AAV2 and the capsid of AAV5).

As referred to herein, the term “adenovirus” refers to a nonenveloped virus with an icosahedral nucleocapsid containing a double stranded DNA of the family Adenoviridae. Over 50 adenoviral subtypes have been isolated from humans and many additional subtypes have been isolated from other mammals and birds. See, e.g., Ishibashi et al., “Adenoviruses of animals,” In The Adenoviruses, Ginsberg, ed., Plenum Press, New York, N.Y., pp. 497-562 (1984); Strauss, “Adenovirus infections in humans,” In The Adenoviruses, Ginsberg, ed., Plenum Press, New York, N.Y., pp. 451-596 (1984). These subtypes belong to the family Adenoviridae, which is currently divided into two genera, namely Mastadenovirus and Aviadenovirus. All adenoviruses are morphologically and structurally similar. In humans, however, adenoviruses show diverging immunological properties and are, therefore, divided into serotypes. Two human serotypes of adenovirus, namely AV2 and AVS, have been studied intensively and have provided the majority of general information about adenoviruses.

In embodiments, the methods of production of viral vector provided herein produce a lentivirus vector.

As referred herein, the term “lentivirus vector” refers to an enveloped virus with a small spherical shape containing two single stranded RNA molecules belonging to the family Retroviridae. Lentiviruses contain gag, pol, and env genes and are further distinguished from other retrovirus family members by having two regulatory genes, that and rev. Lentivirus vectors are widely known in the art as useful tools in molecular biology to induce expression of genes of interest in cultured cells and animal tissues.

In embodiments, the methods of production of viral vector provided herein produce a retrovirus vector.

As referred herein, the term “retrovirus” refers to one or more members of the family Retroviridae, which are enveloped viruses with a small spherical shape containing two single stranded RNA molecules. Retroviruses convert their RNA molecules into DNA, which is then integrated into the host genome of the infected cell. Retrovirus-based vectors are well-known in the field of gene therapy for cancer treatment where immune cells are re-programmed to target and destroy cancer cells.

In embodiments, the methods of production of viral vector provided herein produce a baculovirus vector.

As referred herein, the term “baculovirus” refers to one or more members of the family Baculoviridae that are rod-shaped viruses containing a circular dsDNA and are known to primarily infect and replicate within insect larvae. Baculovirus expression vector systems are well-established and highly useful for the production of proteins in eukaryotic cells (Summers et al., 2006).

In additional embodiments, the methods suitably utilize an insect cell as the viral producer cell. As referred to herein, an “insect cell” suitably refers to cells that originate from insects such as but not limited to members of the lepidopteran order used for expression and manufacture of proteins and/or baculovirus vector production.

In embodiments, the methods suitably utilize an Sf9 cell. As referred here, an “Sf9 cell” is an insect cell line derived from pupal ovarian tissue of the worm Spodoptera frugiperda, commonly used for expression and manufacture of proteins and/or baculovirus vector production.

In embodiments, an amount of viral vector produced by the methods described herein is at least about 10¹⁰ viral vectors. For example, the amount of viral vectors produced by the methods described herein is at least about 10¹⁰ viral vectors, or at least about 10¹¹ viral vectors, or at least about 10¹² viral vectors, or at least about 10¹³ viral vectors, or at least about 10¹⁴ viral vectors, or about 10¹⁰-10¹⁴ viral vectors, or about 10¹⁰-10¹³ viral vectors, or about 10¹⁰-10¹² viral vectors, or about 10¹⁰, about 10¹¹, about 10¹², or about 10¹³ viral vectors.

In embodiments, the methods described herein are for production of an adeno-associated virus (AAV) viral vector. Such processes suitably comprise introducing an engineered mammalian AAV viral producer into a fully enclosed cell engineering system. As used herein, a “viral producer cell” refers to a cell that includes, integrated into its genome, or more viral helper or viral packaging genes. AAV viral producer cells suitably include, integrated into their genome, an adenovirus helper gene comprising E2A and E4Orf6 genes under control of a first derepressible promoter. Exemplary engineered mammalian AAV viral producer cells suitably utilized in the methods to produce an AAV viral vector are described in detail in U.S. provisional applications 62/783,589 and 62/866,092 which are hereby incorporated by reference in their entireties.

As described herein, suitably the mammalian AAV viral producer cells utilized in the methods include a nucleic acid molecule encoding a viral helper gene. Viral helper genes include various adenoviral virus genes, herpes virus genes and bocavirus genes (see, e.g., Guido et al., “Human bocavirus: Current knowledge and future challenges,” World J. Gateroenterol 22:8684-8697, the disclosure of which is incorporated by reference herein in its entirety). In exemplary embodiments, the viral helper gene is an adenovirus helper gene. As referred to herein, the term “adenovirus helper gene” or “AV helper gene” refers to a gene that is composed of one or more nucleic acid sequences derived from one or more adenovirus subtypes or serotypes that contributes to Adeno-associated virus replication and packaging. In some embodiments, the Adenovirus helper gene is E1A, E1B, E2A, E4 (including E4Orf6), VA, or a combination thereof or any other adenovirus helper gene. In exemplary embodiments, the adenovirus helper gene comprises both E2A and E4Orf6 genes. Suitably, an internal ribosome entry site (IRES) element is included between the E2A and E4Orf6 genes. The IRES element initiates translation of the E4Orf6 gene after the E2A gene in a single expression cassette, providing stability to the construct. Such viral helper genes can also be added to a packaging cell by introduction using transient transfection.

In further embodiments, the method of automated production of an AAV viral vector comprises an engineered mammalian viral producer cell that contains an AAV gene comprising Rep and Cap genes under control of a promoter. These AAV genes can also be transiently transfected into viral packaging cells.

As referred to herein, the term “Rep” gene refers to the art-recognized region of the AAV genome which encodes the replication proteins of the virus which are collectively required for replicating the viral genome, or functional homologues thereof such as the human herpesvirus 6 (HHV-6) rep gene which is also known to mediate AAV-2 DNA replication. Thus, the rep coding region can include the genes encoding for AAV Rep78 and Rep68 (the “long forms of Rep”), and Rep52 and Rep40 (the “short forms of Rep”), or functional homologues thereof. The rep coding region, as used herein, can be derived from any viral serotype, such as the AAV serotypes described herein. The region need not include all wild-type genes but may be altered, (e.g., by insertion, deletion or substitution of nucleotides), so long as the rep genes present provide for sufficient integration functions when expressed in a suitable target cell. See, e.g. Muzyczka, N., Current Topics in Microbiol. and Immunol. 158:97-129 (1992); and Kotin, R. M., Human Gene Therapy 5:793-801 (1994).

As referred to herein, the term “Cap” gene refers to the art-recognized region of the AAV genome which encodes the capsid proteins of the virus. Illustrative (non-limiting) examples of these capsid proteins are the AAV capsid proteins VP1, VP2, and VP3. Cap genes used in this disclosure can come from any AAV serotype or a combination of AAV serotypes.

In further embodiments, the method suitably comprises expanding the transduced and producing the AAV viral vector and subsequently isolating the viral vector.

In embodiments, the steps of the method are performed in a closed and automated process.

In embodiments, the engineered mammalian AAV viral producer cell utilized in the methods of production is a mammalian cell culture, which, in some embodiments, suitably is a suspension culture. Exemplary mammalian cells include CHO cells or human cells, including HEK cells.

In embodiments, the method of automated production of an AAV viral vector produces at least about 10¹⁰ viral vectors. For example, the amount of AAV viral vectors produced by the methods described herein is at least about 10¹⁰ AAV viral vectors, or at least about 10¹¹ AAV viral vectors, or at least about 10¹² AAV viral vectors, or at least about 10¹³ AAV viral vectors, or at least about 10¹⁴ AAV viral vectors, or about 10¹⁰-10¹⁴ AAV viral vectors, or about 10¹⁰-40¹³ AAV viral vectors, or about 10¹⁰-10¹² AAV viral vectors, or about 10¹⁰, about 10¹¹, about 10¹², or about 10¹³ AAV viral vectors.

In additional exemplary embodiments, the method disclosed is a method for automated production of a lentiviral vector, comprising introducing an engineered mammalian lentiviral vector producer cell into a fully enclosed cell engineering system. A packaging cell can also be used to produce lentiviral vectors. Exemplary methods of producing lentivirus can be found in U.S. Provisional Patent Application No. 62/890,904, filed Aug. 23, 2019, and 62/949,848, filed Dec. 18, 2019, the disclosures of each of which are incorporated by reference herein in their entireties.

As used herein a “lentiviral vector producer cell” refers to a cell that contains, integrated into its genome, the elements required to produce a lentiviral vector. These elements can also be introduced into a packaging cell to produce a lentiviral vector.

In embodiments, the method utilizes a lentiviral vector producer cell including integrated into its genome a lentiviral regulator of expression of virion proteins (REV) gene under control of a first promoter, a lentiviral envelope gene under control of a second promoter, and a lentiviral group specific antigen (GAG) gene and a lentiviral polymerase (POL) gene both under control of a third promoter. In suitable embodiments, the nucleic acid sequence is flanked on both the 5′ and 3′ ends by sequences resulting from the recombination of transposon-specific inverted terminal repeats (ITRs).

As disclosed herein, the lentiviral regulator of expression of virion proteins (REV) is an RNA-binding protein that promotes late phase gene expression. It is also important for the transport of the unspliced or singly-spliced mRNAs, which encode viral structural proteins, from the nucleus to the cytoplasm.

The lentiviral envelope (ENV) gene, suitably a Vesicular Somatitis Virus Glycoprotein (VSV-G) gene, encodes a polyprotein precursor which is cleaved by a cellular protease into the surface (SU) envelope glycoprotein gp120 and the transmembrane (TM) glycoprotein gp41.

GAG encodes a polyprotein that is translated from an unspliced mRNA which is then cleaved by the viral protease (PR) into the matrix protein, capsid, and nucleocapsid proteins. The lentiviral polymerase (POL) is expressed as a GAG-POL polyprotein as a result of ribosomal frameshifting during GAG mRNA translation, and encodes the enzymatic proteins reverse transcriptase, protease, and integrase. These three proteins are associated with the viral genome within the virion. Suitably the GAG gene is an HIV GAG gene and the POL gene is an HIV POL gene.

In suitable embodiments, the expression cassette is flanked on both the 5′ and 3′ ends by transposon-specific inverted terminal repeats (ITR).

Exemplary promoters for use in the lentiviral vector-producing cells are known in the art and include derepressible promoters, and suitably the expression cassette further encodes a repressor element of the first, second and third derepressible promoters. In embodiments, the derepressible promoters comprises a functional promoter and a tetracycline operator sequence (TetO), and the repressor element is a tetracycline repressor protein, as described herein.

In further embodiments, the method to produce a lentiviral vector includes transducing the mammalian lentiviral vector producer cell with a vector encoding a gene of interest. In embodiments, the gene of interest is a gene of therapeutic interest.

In further embodiments, the method includes activation of the first, second, and third promoters within the lentiviral vector producer cell and expanding the transduced viral producer cell.

In further embodiments, the method includes suitably isolating the produced lentiviral vector. Methods for isolated produced viral vectors are described herein.

In exemplary embodiments, the method is performed in a closed an automated process.

As described herein, suitably the automated method utilizes a mammalian cell that is a mammalian cell culture, and in embodiments is a suspension culture. Exemplary cells include human cells such as HEK293 or HEK293T cells.

In embodiments, the method of automated production of a lentiviral viral vector produces at least about 10¹⁰ viral vectors. For example, the amount of lentiviral vectors produced by the methods described herein is at least about 10¹⁰ lentiviral vectors, or at least about 10¹¹ lentiviral vectors, or at least about 10¹² lentiviral vectors, or at least about 10¹³ lentiviral vectors, or at least about 10¹⁴ lentiviral vectors, or about 10¹⁰-10¹⁴ lentiviral vectors, or about 10¹⁰-10¹³ lentiviral vectors, or about 10¹⁰-10¹² lentiviral vectors, or about 10¹⁰, about 10¹¹, about 10¹², or about 10¹³1entiviral vectors.

In embodiments, the steps of the method are performed in a closed and automated process, and suitably include monitoring with one or more of a temperature sensor, a pH sensor, a glucose sensor, a lactose sensor, an oxygen sensor, a carbon dioxide sensor, and an optical density sensor, and automatically adjusting one or more of a temperature, a pH level, a glucose level, a lactose level, an oxygen level, a carbon dioxide level, and an optical density.

Also provided herein are methods of treating a mammalian subject, suitably a human subject, with an AAV or lentiviral vector produced according to the various methods described herein. Suitably, the methods are used to treat a human subject with a gene of interest, including a gene of therapeutic interest. Administration to a human subject can include, for example, inhalation, injection, or intravenous administration, as well as other administration methods known in the art.

### Additional Exemplary Embodiments

Embodiment 1 is a method automated production of a viral vector, comprising: introducing an engineered viral producer cell into a fully enclosed cell engineering system; transducing the engineered viral producer cell with a vector encoding a gene of interest to produce a transduced viral producer cell; expanding the transduced viral producer cell and producing the viral vector within the transduced viral producer cell; transferring the expanded producer cell to a downstream processing module; and isolating the viral vector; and purifying the viral vector, wherein the elements are performed in a closed and automated process.

Embodiment 2 includes the method of embodiment 1, wherein the engineered viral producer cell is a mammalian cell.

Embodiment 3 includes the method of embodiment 2, wherein the mammalian cell is a mammalian cell culture.

Embodiment 4 includes the method of embodiment 3, wherein the mammalian cell culture is a suspension culture.

Embodiment 5 includes the method of any of embodiments 1-4, wherein the viral vector is an adeno-associated virus (AAV) vector.

Embodiment 6 includes the method of any of embodiments 1-4, wherein the viral vector is a lentivirus vector.

Embodiment 7 includes the method of any of embodiments 1-4, wherein the viral vector is a retrovirus vector.

Embodiment 8 includes the method of any of embodiments 1-4, wherein the viral vector is a baculovirus vector.

Embodiment 9 includes the method of any of embodiments 2-8, wherein the mammalian cell is a Chinese hamster ovary (CHO) cell.

Embodiment 10 includes the method of any of embodiments 2-8, wherein the mammalian cell is a human cell.

Embodiment 11 includes the method of embodiment 10, wherein the human cell is a human embryonic kidney (HEK) cell.

Embodiment 12 includes the method of embodiment 10, wherein the human cell is a HEK293T cell.

Embodiment 13 includes the method of embodiment 1, wherein the engineered producer cell is an insect cell.

Embodiment 14 includes the method of embodiment 1, wherein the engineered producer cell is an Sf9 cell.

Embodiment 15 includes the method of embodiment 1, wherein an amount of viral vectors produced is at least about 1010 viral vectors.

Embodiment 16 includes the method of any of embodiments 1-15, wherein the closed and automated process comprises: monitoring with one or more of a temperature sensor, a pH sensor, a glucose sensor, a lactose sensor, an oxygen sensor, a carbon dioxide sensor, and an optical density sensor, and automatically adjusting one or more of a temperature, a pH level, a glucose level, a lactose level, an oxygen level, a carbon dioxide level, and an optical density.

Embodiment 17 includes the method of any of embodiments 1-16, wherein the transducing comprises viral infection, electroporation, liposome transfection, or membrane disruption.

Embodiment 18 includes the method of any of embodiments 1-17, wherein the isolating comprises passing the expanded producer cells through an elution column.

Embodiment 19 includes the method of any of embodiments 1-18, wherein the purifying comprises membrane polishing.

Embodiment 20 includes the method of any of embodiments 1-19, further comprising formulating the viral vector.

Embodiment 21 is a method for automated production of a viral vector, comprising: introducing a packaging cell into a fully enclosed cell engineering system; transducing the packaging cell with one or more vectors encoding a viral helper gene, a viral packing gene, and a gene of interest to produce a transduced cell; expanding the transduced cell and producing the viral vector within the transduced cell; transferring the expanded cell to a downstream processing module; and isolating the viral vector; and purifying the viral vector, wherein the elements are performed in a closed and automated process.

Embodiment 22 includes the method of embodiment 21, wherein the packaging cell is a mammalian cell.

Embodiment 23 includes the method of embodiment 22, wherein the mammalian cell is a mammalian cell culture.

Embodiment 24 includes the method of embodiment 23, wherein the mammalian cell culture is a suspension culture.

Embodiment 25 includes the method of any of embodiments 21-24, wherein the viral vector is an adeno-associated virus (AAV) vector.

Embodiment 26 includes the method of any of embodiments 21-24, wherein the viral vector is a lentivirus vector.

Embodiment 27 includes the method of any of embodiments 21-24, wherein the viral vector is a retrovirus vector.

Embodiment 28 includes the method of any of embodiments 21-24, wherein the viral vector is a baculovirus vector.

Embodiment 29 includes the method of any of embodiments 22-28, wherein the mammalian cell is a Chinese hamster ovary (CHO) cell.

Embodiment 30 includes the method of any of embodiments 22-28, wherein the mammalian cell is a human cell.

Embodiment 31 includes the method of embodiment 30, wherein the human cell is a human embryonic kidney (HEK) cell.

Embodiment 32 includes the method of embodiment 30, wherein the human cell is a HEK293T cell.

Embodiment 33 includes the method of embodiment 21, wherein the packaging cell is an insect cell.

Embodiment 34 includes the method of embodiment 21, wherein the packaging cell is an Sf9 cell.

Embodiment 35 includes the method of embodiment 21, wherein an amount of viral vectors produced is at least about 1010 viral vectors.

Embodiment 36 includes the method of any of embodiments 21-35, wherein the closed and automated process comprises: monitoring with one or more of a temperature sensor, a pH sensor, a glucose sensor, a lactose sensor, an oxygen sensor, a carbon dioxide sensor, and an optical density sensor, and automatically adjusting one or more of a temperature, a pH level, a glucose level, a lactose level, an oxygen level, a carbon dioxide level, and an optical density.

Embodiment 37 includes the method of any of embodiments 21-36, wherein the transducing comprises viral infection, electroporation, liposome transfection, or membrane disruption.

Embodiment 38 includes the method of any of embodiments 21-37, wherein the isolating comprises passing the expanded producer cells through an elution column.

Embodiment 39 includes the method of any of embodiments 21-38, wherein the purifying comprises membrane polishing.

Embodiment 40 includes the method of any of embodiments 21-39, further comprising formulating the viral vector.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A method for automated production of a viral vector, comprising:
(a) introducing an engineered viral producer cell into a fully enclosed cell engineering system;
(b) transducing the engineered viral producer cell with a vector encoding a gene of interest to produce a transduced viral producer cell;
(c) expanding the transduced viral producer cell and producing the viral vector within the transduced viral producer cell;
(d) transferring the expanded producer cell to a downstream processing module; and
(e) isolating the viral vector; and
(f) purifying the viral vector,
wherein (a) through (e) are performed in a closed and automated process.

2. The method of claim 1, wherein the engineered viral producer cell is a mammalian cell.

3. (canceled)

4. (canceled)

5. The method of claim 1, wherein the viral vector is an adeno-associated virus (AAV) vector, a lentivirus vector, a retrovirus vector or a baculovirus vector.

6. (canceled)

7. (canceled)

8. (canceled)

9. The method of claim 2, wherein the mammalian cell is a Chinese hamster ovary (CHO) cell.

10. (canceled)

11. The method of claim 2, wherein the humanmammalian cell is a human embryonic kidney (HEK) cell.

12. (canceled)

13. The method of claim 1, wherein the engineered producer cell is an insect cell.

14. (canceled)

15. (canceled)

16. The method of claim 1, wherein the closed and automated process comprises:
(a) monitoring with one or more of a temperature sensor, a pH sensor, a glucose sensor, a lactose sensor, an oxygen sensor, a carbon dioxide sensor, and an optical density sensor, and
(b) automatically adjusting one or more of a temperature, a pH level, a glucose level, a lactose level, an oxygen level, a carbon dioxide level, and an optical density.

17. The method of claim 16, wherein the transducing comprises viral infection, electroporation, liposome transfection, or membrane disruption.

18. The method of claim 17, wherein the isolating comprises passing the expanded producer cells through an elution column.

19. The method of claim 17, wherein the purifying comprises membrane polishing.

20. (canceled)

21. A method for automated production of a viral vector, comprising:
(a) introducing a packaging cell into a fully enclosed cell engineering system;
(b) transducing the packaging cell with one or more vectors encoding a viral helper gene, a viral packing gene, and a gene of interest to produce a transduced cell;
(c) expanding the transduced cell and producing the viral vector within the transduced cell;
(d) transferring the expanded cell to a downstream processing module; and
(e) isolating the viral vector; and
(f) purifying the viral vector, wherein (a) through (e) are performed in a closed and automated process.

22. The method of claim 21, wherein the packaging cell is a mammalian cell.

23. (canceled)

24. (canceled)

25. The method of claim 21, wherein the viral vector is an adeno-associated virus (AAV) vector, a lentivirus vector, a retrovirus vector, or a baculovirus vector.

26. (canceled)

27. (canceled)

28. (canceled)

29. The method of claim 22, wherein the mammalian cell is a Chinese hamster ovary (CHO) cell.

30. (canceled)

31. The method of claim 22, wherein the mammalian cell is a human embryonic kidney (HEK) cell.

32. (canceled)

33. The method of claim 21, wherein the packaging cell is an insect cell.

34. (canceled)

35. (canceled)

36. The method of claim 21, wherein the closed and automated process comprises:
(c) monitoring with one or more of a temperature sensor, a pH sensor, a glucose sensor, a lactose sensor, an oxygen sensor, a carbon dioxide sensor, and an optical density sensor, and
(d) automatically adjusting one or more of a temperature, a pH level, a glucose level, a lactose level, an oxygen level, a carbon dioxide level, and an optical density.

37. The method of claim 36, wherein the transducing comprises viral infection, electroporation, liposome transfection, or membrane disruption.

38. The method of claim 37, wherein the isolating comprises passing the expanded producer cells through an elution column.

39. The method of claim 37, wherein the purifying comprises membrane polishing.

40. (canceled)
